(19) United States
(12) Patent Application Publication
(10) Pub. No.: US 2011/0172478 A1
(43) Pub. Date: Jul. 14, 2011

(54) CATALYST FOR AROMATIZATION OF LOWER HYDROCARBON, AND PROCESS FOR PRODUCTION OF AROMATIC COMPOUND

(75) Inventors: Hongtao Ma, Tokyo (JP); Yuji Ogawa, Saitama (JP)

(73) Assignee: MEIDENSHA CORPORATION

(21) Appl. No.: 13/058,413

(22) PCT Filed: Jun. 18, 2009
(86) PCT No.: PCT/JP2009/061070

§ 371 Date: Feb. 10, 2011

(30) Foreign Application Priority Data
Aug. 12, 2008 (JP) 2008-207757

(51) Int. Cl. C07C 2/00; B01J 21/06; B01J 29/48

(52) U.S. Cl. 585/417; 502/255; 502/77

(57) ABSTRACT

[Object] To improve the yield of aromatic hydrocarbon and the stability of active life in a process for producing aromatic compound by using a lower hydrocarbon aromatization catalyst.

[Solving Means] A lower hydrocarbon aromatization catalyst for producing aromatic compound under reaction of lower hydrocarbon has an average crystal diameter of not larger than 500 nm. A catalyst in which molybdenum is carried on ZSM-5 zeolite as metallosilicate is used as an example of the above-mentioned catalyst. Additionally, a process for producing aromatic compound upon contact of the above-mentioned catalyst with a reaction gas containing lower hydrocarbon is provided.

### TECHNICAL FIELD

This invention relates to an advanced use of natural gas, biogas and methane hydrate which contain methane as a main component. Natural gas, biogas, methane hydrate seem to be the most effective energy source for fighting against grovel warming, and attentions to technologies for using them are increasing. Attentions are paid on methane resources as next-generation new organic resources and hydrogen resources for fuel cells because the methane resources make their cleanness effective as they are. This invention relates particularly to a catalytic chemical conversion technology for effectively producing aromatic compounds and high purity hydrogen gas from lower hydrocarbons such as methane, and to a process for producing a catalyst therefor, the aromatic compounds containing as main component benzene and naphthalenes serving as raw materials for chemical products such as plastics.

### BACKGROUND ART

As a process for producing aromatic compounds such as benzene and the like and hydrogen from lower hydrocarbons such as methane and the like, a process for making reaction of lower hydrocarbon in the presence of catalyst is known. As a catalyst for this process, molybdenum carried on ZSM-5 type zeolite seems to be effective (see Non-patent Citation 1). However, it is desired to develop a more excellent catalyst in order to further improve the production efficiency of aromatic compounds and hydrogen gas.

Zeolite as an example of crystalline metallosilicate used as a catalyst for this reaction has usually a solid acid characteristics and a crystal pore diameter of several angstroms (for example, 5 to 6 angstroms in case of ZSM-5) serving as a molecular sieve.

In a reaction in which aromatic hydrocarbon such as benzene or the like is produced from lower hydrocarbon, it is assumed that sequential reactions occur on a catalyst where an active species is carried on metallosilicate thereby producing aromatic hydrocarbon.

Specifically, at the first stage of the sequential reactions, combination reaction is made among lower hydrocarbons such as methane or the like under the action of the carried species or metal species such as molybdenum, tungsten or rhenium or carbides thereof, thereby producing straight-chain hydrocarbon having a carbon number of 2 or more. Next, at the second stage, the above mentioned straight chain hydrocarbon makes its cyclization reaction under the actions of the spaces of pores of metallosilicate serving as a carrier and of Brönsted acid point. In other words, by this reaction, the straight-chain hydrocarbon makes its hydrogenation reaction to be cyclicly formed thereby being converted to aromatic hydrocarbon which is unsaturated cyclic hydrocarbon such as benzene or the like. By the above sequential reactions, aromatic hydrocarbon is produced from lower hydrocarbon.

### PRIOR ART CITATION

### Non-Patent Citation

Non-Patent Citation 1: JOURNAL OF CATALYSIS, 1997, pp. 165, pp. 150-161

### DISCLOSURE OF THE INVENTION

### Problems to be Solved by the Invention

However, in the above-mentioned prior art, in the reaction of producing aromatic hydrocarbon from lower hydrocarbon, the number of inlets of pores at the crystal surface of metallosilicate per unit volume or unit weight, i.e., the pore inlet density becomes a factor of diffusion rate-controlling. Under a diffusion rate-controlled condition, there arises such a problem that the molecular sieve due to the pores of zeolite cannot effectively function so that coking of reaction product occurs with the progression of the reaction on the catalyst thereby lowering the long term stability and the reaction efficiency of the catalyst.

Specifically, zeolite used as the catalyst for this reaction has a solid acid characteristics and the crystal pore diameter of several angstroms serving as a molecular sieve. Usual zeolite has a crystal size of about several μm which is very large as compared with the crystal pore diameter. Accordingly, in case that zeolite is used as a catalyst, the zeolite tends to be put into a diffusion rate-controlling condition where reaction is governed by diffusion of raw material and product within zeolite crystal rather than by its solid acid characteristics. In other words, since the pore inlet density is low, there is a little chance of diffusion and penetration of straight-chain hydrocarbon having a carbon number of 2 or more produced at the first stage of the sequential reactions into pores, so that straight-chain hydrocarbon which cannot reach to cyclization reaction makes its coking at the surface of zeolite thereby providing the factors of lowering the stability of active life of the catalyst and of lowering the yield of aromatic hydrocarbon.

Accordingly, an object of the present invention is to provide a lower hydrocarbon aromatization catalyst which is high in reaction efficiency while reducing the influence of diffusion of substances within pores by using nano-scale zeolite whose zeolite crystal is small-sized.

### Means for Solving the Problems

The lower hydrocarbon aromatization catalyst for attaining the above-mentioned object is a catalyst for producing aromatic compound under reaction of lower hydrocarbon, in which the above-mentioned catalyst is characterized by having an average crystal diameter of not larger than 500 nm.

Additionally, a producing process for aromatic hydrocarbon, according to the present invention is characterized by allowing a reaction gas containing lower hydrocarbon to react with a catalyst including metallosilicate having an average crystal diameter of not larger than 500 nm.

According to the low hydrocarbon aromatization catalyst and a process for producing aromatic compound, of the present invention, the crystal diameter is rendered nano-sized so that the density of pore inlets is increased thereby making it possible to increase the chances of diffusion and penetration of straight-chain hydrocarbon into pores.

An example of the above-mentioned metallosilicate is ZSM-5 zeolite. Additionally, molybdenum may be carried on the above-mentioned metallosilicate.

### Effects of the Invention

Hence, according to the present invention mentioned above, in connection with the aromatic compound producing process using the lower hydrocarbon aromatization catalyst, the yield of aromatic hydrocarbon and the active life stability of the catalyst are improved.

### BRIEF DESCRIPTION OF THE DRAWINGS

FIG. 1 shows variations per hour, of benzene yields (%) in aromatization reactions of lower hydrocarbon by lower hydrocarbon aromatization catalysts according to an embodiment of the present invention.

### MODE FOR CARRYING OUT THE INVENTION

A lower hydrocarbon aromatization catalyst according to an embodiment of the present invention can be obtained by causing a precursor containing molybdenum to be carried on metallosilicate.

Examples of metallosilicate to be used for the catalyst are molecular sieve 5A (UTA) forming a porous body containing silica and alumina, faujasite (NaY), and aluminosilicate such as NaX, ZSM-5, H-ZSM-5. Another example of metallosilicate to be used for the catalyst is a porous carrier such as ALPO-5, VPI-5 and the like containing phosphoric acid as main component and is a zeolite carrier characterized by micro-pores or channels having pore diameters of 0.6 nm to 1.3 nm. A further example of metallosilicate to be used for the catalyst is a meso-pore porous carrier such as FSM-16, MCM-41 and the like containing silica as a main component and partly alumina as a component and being characterized by cylindrical pores (channels) or meso-pores (pore diameter: 1 nm to 10 nm).

Examples of the precursor containing molybdenum are ammonium paramolybdate, phosphomolybdic acid, 12 silicomolybdic acid, and halogenide thereof such as chloride, bromide and the like, mineral acid salt thereof such as nitrate, sulfate, phosphate and the like, carbonate thereof, and carboxylate thereof such as oxalate and the like, and the like thereof.

A general method for causing molybdenum to be carried on metallosilicate is as follows: A metallosilicate carrier is impregnated with an aqueous solution of the above-mentioned precursor containing molybdenum so that the precursor is carried on the carrier. Thereafter, the impregnated carrier is subjected to a heating treatment in the air.

A concrete example of this carrying method is as follows: A metallosilicate carrier is impregnated with and carries ammonium molybdate. After drying, the impregnated carried is subjected to a heating treatment at 250° C. to 800° C., preferably 400° C. to 700° C. in air stream, thereby producing a metallosilicate catalyst.

The catalyst to be used in the present invention may be formed into pellets or an extruded product, upon binder such as silica, alumina and/or clay being added.

Here, examples of lower hydrocarbon used in the present invention are methane, and saturated or unsaturated hydrocarbon having carbon numbers of 2 to 6. It is preferable that a gas to be reacted contains at least 50% by weight, preferably at least 70% by weight of methane. The gas may contain saturated or unsaturated hydrocarbons having carbon numbers of 2 to 6 in addition to methane. Examples of the saturated or unsaturated hydrocarbons having carbon numbers of 2 to 6 are ethane, ethylene, propane, propylene, n-butane, isobutane, n-butene, isobutene, and the like.

Aromatization reaction of lower hydrocarbon in a process for producing aromatic hydrocarbon and hydrogen from lower hydrocarbon according to the present invention can be accomplished by a batch mode or a flow-mode. Particularly, it is preferable to accomplish the reaction by the flow-mode using a fixed bed, a moving bed, a fluidized bed, or the like.

A catalytic reaction is made by contacting the raw material of lower hydrocarbon with the catalyst at a reaction temperature of 300° C. to 900° C., preferably 450° C. to 800° C., and at a reaction pressure of 0.01 MPa to 1 MPa, preferably 0.1 MPa to 0.7 MPa.

The present invention will be discussed more in detail with reference to Examples. An average crystal diameter is determined by calculating an average value of particles randomly selected from an electron microscopic picture. A benzene yield is defined by the following equation (1):

Benzene yield (%)={(quantity of benzene produced)/(quantity of methane supplied to a methane reforming reaction)}×100 (1)

### Comparative Example 1

Commercially available H-type ZSM-5 zeolite (SiO₂/Al₂O₃=28) having an average crystal diameter of 1 μm in an amount of 400 g was mixed as a metallosilicate carrier in an aqueous solution which was prepared by dissolving 44.2 g of ammonium molybdate in 1500 ml of ion-exchanged water. The aqueous solution containing the carrier was stirred at room temperature for 3 hours so that the carrier was impregnated with and carried ammonium molybdate. After dried, the carrier was calcined at 550° C. for 8 hours thereby obtaining a catalyst.

### Example 1

The same preparation as in Comparative Example 1 was carried out with the exception that zeolite having a different average crystal diameter was used. Specifically, 400 g of commercially available H-type ZSM-5 zeolite (SiO₂/Al₂O₃=28) having an average crystal diameter of 70-80 nm was mixed as a metallosilicate carrier in an aqueous solution which was prepared by dissolving 44.2 g of ammonium molybdate in 1500 ml of ion-exchanged water. The aqueous solution containing the carrier was stirred at room temperature for 3 hours so that the carrier was impregnated with and carried ammonium molybdate. After dried, the carrier was calcined at 550° C. for 8 hours thereby obtaining a catalyst.

### Example 2

The same preparation as in Comparative Example 1 was carried out with the exception that zeolite having a different average crystal diameter was used. Specifically, 400 g of commercially available H-type ZSM-5 zeolite (SiO₂/Al₂O₃=28) having an average crystal diameter of 500 nm was mixed as a metallosilicate carrier in an aqueous solution which was prepared by dissolving 44.2 g of ammonium molybdate in 1500 ml of ion-exchanged water. The aqueous solution containing the carrier was stirred at room temperature for 3 hours so that the carrier was impregnated with and carried ammonium molybdate. After dried, the carrier was calcined at 550° C. for 8 hours thereby obtaining a catalyst.

By using the catalysts prepared respectively under the conditions of Examples 1 and 2 and Comparative Example 1, aromatic compounds were produced from lower hydrocarbons, thereby evaluating catalyst performances of the catalysts. The index of the catalyst performance to be evaluated is a rate of benzene to lower hydrocarbon flowing through the catalyst.

A reaction test for evaluating the catalyst performance of each catalyst was carried out under a reaction test condition where a methane reaction temperature was 780° C., a pressure was 0.3 MPa, and a weight hourly space velocity (WHSV) was 3000 ml/g/h. A reaction gas used as the raw material of lower hydrocarbon had a composition including 90% of methane and 10% of argon. In order to carry out the reaction test, a pretreatment of the catalyst was made in which the temperature of the catalyst was raised to 550° C. in the stream of air and kept for 2 hours; and thereafter, the temperature of the catalyst was raised to 700° C. upon replacing air with a pretreatment gas containing 20% of methane and 80% of hydrogen and kept for 3 hours. Thereafter, the pretreatment gas was replaced with the reaction gas, and the temperature of the catalyst was raised to 780° C. to accomplish the reaction, thus confirming the catalyst performance of the catalyst upon evaluating the activity of the catalyst.

Hydrogen, argon and methane were analyzed by an apparatus TCD-GC, and aromatic compounds such as benzene, toluene, xylene, naphthalene and the like were analyzed by an apparatus FID-GC.

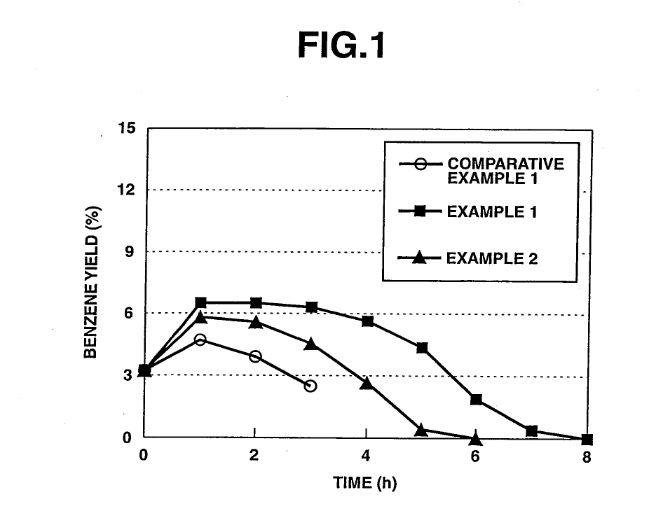

Analysis results are shown in Table 1 and FIG. 1. Table 1 shows the benzene yields (%) with the respective catalysts, obtained when 3 hours lapsed after the initiation of the reaction. FIG. 1 shows variations per hour, of the benzene yields with the respective catalysts.

TABLE 1

| | Average crystal | Benzene |
|---|---|---|
| | diameter | yield |
| Comparative | Average crystal | 2.5% |
| Example 1 | diameter: 1 micrometer | |
| Example 1 | Average crystal | 6.7% |
| | diameter: 70-80 nm | |
| Example 2 | Average crystal | 4.6% |
| | diameter: 500 nm | |

As apparent from Table 1, the benzene yield of Comparative Example 1 is 2.5%, whereas the benzene yield of Example 1 is 6.7% and the benzene yield of Example 2 is 4.6%, so that the benzene yield is improved as the crystal diameter is smaller. Additionally, as shown in FIG. 1, with reference to the variations per hour, of the benzene yields, it is revealed that the higher benzene yields are kept as the crystal diameter is smaller.

As described above, according to the lower hydrocarbon aromatization catalyst of the present invention, by rendering the crystal diameter nano-sized, the density of pore inlets becomes high so as to increasing the chances of diffusion and penetration of straight-chain hydrocarbon into pores. Accordingly, cyclization reaction can smoothly progress thereby suppressing a decrease in number of the pore inlets, due to corking as a side reaction.

Specifically, since the present invention is applied to the sequential reaction, there is a fear that a substance produced at the first stage of the reaction becomes a cause for lowering the activity of the catalyst. In view of this, according to the present invention, chances for reaction to the second stage are increased thereby suppressing occurrence of corking so as to improve the stability of active life of the catalyst.

As a result, in connection with the reaction of aromatization of lower hydrocarbon under the action of the lower hydrocarbon aromatization catalyst, the yield of aromatic hydrocarbon and the active life stability of the catalyst are improved.

1-4. (canceled)

5. A lower hydrocarbon aromatization catalyst for producing aromatic compound by contacting with methane, characterized by comprising:
metallosilicate having an average crystal diameter of not larger than 500 nm and carrying molybdenum.

6. A lower hydrocarbon aromatization catalyst as claimed in claim 5, characterized in that the metallosilicate is ZSM-5 zeolite.

7. A process for producing an aromatic compound, characterized by comprising:
allowing methane to contact with a catalyst including metallosilicate having an average crystal diameter of not larger than 500 nm and carrying molybdenum.
